(19) United States
(12) Patent Application Publication
(10) Pub. No.: US 2013/0181100 A1
(43) Pub. Date: Jul. 18, 2013

(54) TRANSFORMABLE INTRAVENOUS POLE

(71) Applicants: Peter B. Blankenship, Anoka, MN (US); Samuel A. Blankenship, Anoka, MN (US); Brent G. Norman, Champlin, MN (US)

(72) Inventors: Peter B. Blankenship, Anoka, MN (US); Samuel A. Blankenship, Anoka, MN (US); Brent G. Norman, Champlin, MN (US)

(73) Assignee: Streamline, Inc., Minneapolis, MN (US)

(21) Appl. No.: 13/724,774

(22) Filed: Dec. 21, 2012

Related U.S. Application Data
(63) Continuation-in-part of application No. 13/465,617, filed on May 7, 2012.
Application No. 13/465,617 is a continuation-in-part of application No. 11/948,536, filed on Nov. 30, 2007, now Pat. No. 7,918,422.
Application No. 11/948,536 is a continuation-in-part of application No. 11/711,478, filed on Feb. 27, 2007, now Pat. No. 7,497,407.

(51) Int. Cl. A61M 5/14

(52) U.S. Cl. CPC A61M 5/1415
USPC 248/129

(57) ABSTRACT

An intravenous (IV) pole system for supporting medical equipment having a mast engaged to a base, the base having legs to support the mast and a lifting mechanism engaged to the base and the mast for moving the base along the mast. Each of the legs can include at least one wheel. The lifting mechanism is constructed to position the legs in an extended configuration and a retracted configuration. The lifting mechanism is further constructed to raise the legs and wheels relative to the base and relative to a surface. The lifting mechanism is configured to position the wheels proximate to the mast in the retracted configuration.

### CROSS-REFERENCE TO RELATED APPLICATIONS

This application is a continuation-in-part of, and claims priority to, reissue application with Ser. No. 13/465,617 filed on May 7, 2012, of application Ser. No. 11/948,536 now U.S. Pat. No. 7,918,422 which is also a continuation-in-part of, and claims priority to, Ser. No. 11/711,478 filed on Feb. 27, 2007 which is now U.S. Pat. No. 7,497,407, which are herein incorporated by reference in their entirety, which claims priority to provisional patent application 60/777,467.

### STATEMENT REGARDING FEDERALLY SPONSORED RESEARCH

Not Applicable

### BACKGROUND

For many years, patients needing intravenous (IV) fluid transfusions have been able to be moved by a patient transportation apparatus such as a wheelchair, wheeled bed, stretcher, gurney or the like while receiving such transfusions by the use of mobile IV poles. These IV poles, however, suffer from a number of drawbacks.

One unsatisfactory form of a mobile IV pole is a pole permanently attached to, and using out of, the patient transportation device such as that described in published US Patent Application 2006/0243500A1. This form imposes significant burdens on hospital staff as each time the patient is moved, pumps and fluid bags must both be transferred onto the transportation device before the patient is moved, and then again the pumps and fluid bags must be transferred off of the transportation device once the patient arrives at their destination. The repeated transfer of bags and pumps increases the risks of bags or pumps being dropped leading to wasted medicines needing replacement and wasted environmental services cleaning up spills as well as damage occurring to expensive pumps and equipment. Similarly, the permanently attached pole makes the patient transportation device bulky causing awkward and difficult movement, storage, and maintenance. In the case of beds, permanently attached poles render the beds particularly bulky, and difficult to maneuver. Other problems relate to difficulty in linen changes. Further, Most seriously of all, constant removal and re-attachment of IV bags and pumps increases the risks of IV leads being strained or pulled entirely from the patient's body, complicating a patient's treatment and potentially putting the individual at risk of infection or improper treatment.

Another unsatisfactory form of mobile IV pole is a free standing wheeled pole that is moved alongside the patient transfer apparatus such as that described in published US Patent Application 2006/0222341A1. This device unfortunately also imposes significant burdens on hospital staff. In this device one hand must be used to push the patient transfer apparatus and another to simultaneously move the IV pole. Because patient transfer apparatuses may be heavy, and not designed for one handed pushing, repeatedly utilizing one person to simultaneously move both the pole and the transfer apparatus causes significant strain, which often results in back and sprain injuries in medical personnel. Also, such pushing increases the risk of injury to a patient in that the pole may tip over onto the patient or that the patient transfer apparatus may strike walls or objects and aggravate an injury. The alternative of utilizing multiple medical personnel to transport a single patient is inefficient and cost prohibitive in an era of scarce nurses and other medical personnel.

For at least these reasons there is a need for an improved IV pole. The art referred to and/or described above is not intended to constitute an admission that any patent, publication or other information referred to herein is “prior art” with respect to this invention. In addition, this section should not be construed to mean that a search has been made or that no other pertinent information as defined in 37 CFR §1.56(a) exists.

All US patents and applications and all other published documents mentioned anywhere in this application are incorporated herein by reference in their entirety. Without limiting the scope of the invention a brief summary of some of the claimed embodiments of the invention is set forth below. Additional details of the summarized embodiments of the invention and/or additional embodiments of the invention may be found in the Detailed Description below.

### SUMMARY

The invention generally relates to an IV pole system for supporting medical equipment comprising a base, a mast and a lifting mechanism. Specifically, the IV pole is constructed and arranged to position a plurality of legs in either an extended configuration and in a retracted configuration. The invention also relates to a method of retracting and extending a plurality of legs of the base of an IV pole system.

This and other aspects of the invention are described in more detail in the accompanying description and drawings.

### BRIEF DESCRIPTION OF THE SEVERAL VIEWS OF THE DRAWINGS

The invention is best understood from the following detailed description when read in connection with accompanying drawings, in which:

FIG. 1 is a perspective view of a self retracting IV pole in the expanded configuration supported by a bed mount.

FIG. 2 is another perspective view of a self retracting IV pole in the expanded configuration supported by a bed mount.

FIG. 3 is a perspective view of a self retracting IV pole in the expanded configuration detached from a bed mount.

FIG. 4A is a perspective view of a self retracting IV pole in the retracted configuration supported by a bed mount.

FIG. 4B is a side elevation view of a self retracting IV pole in the retracted configuration supported by a bed mount.

FIG. 5A is a perspective view of a self retracting IV pole in the retracted configuration supported by a bed mount coupled to a bed.

FIG. 5B is a perspective view of a bed mount coupled to a bed.

### DETAILED DESCRIPTION

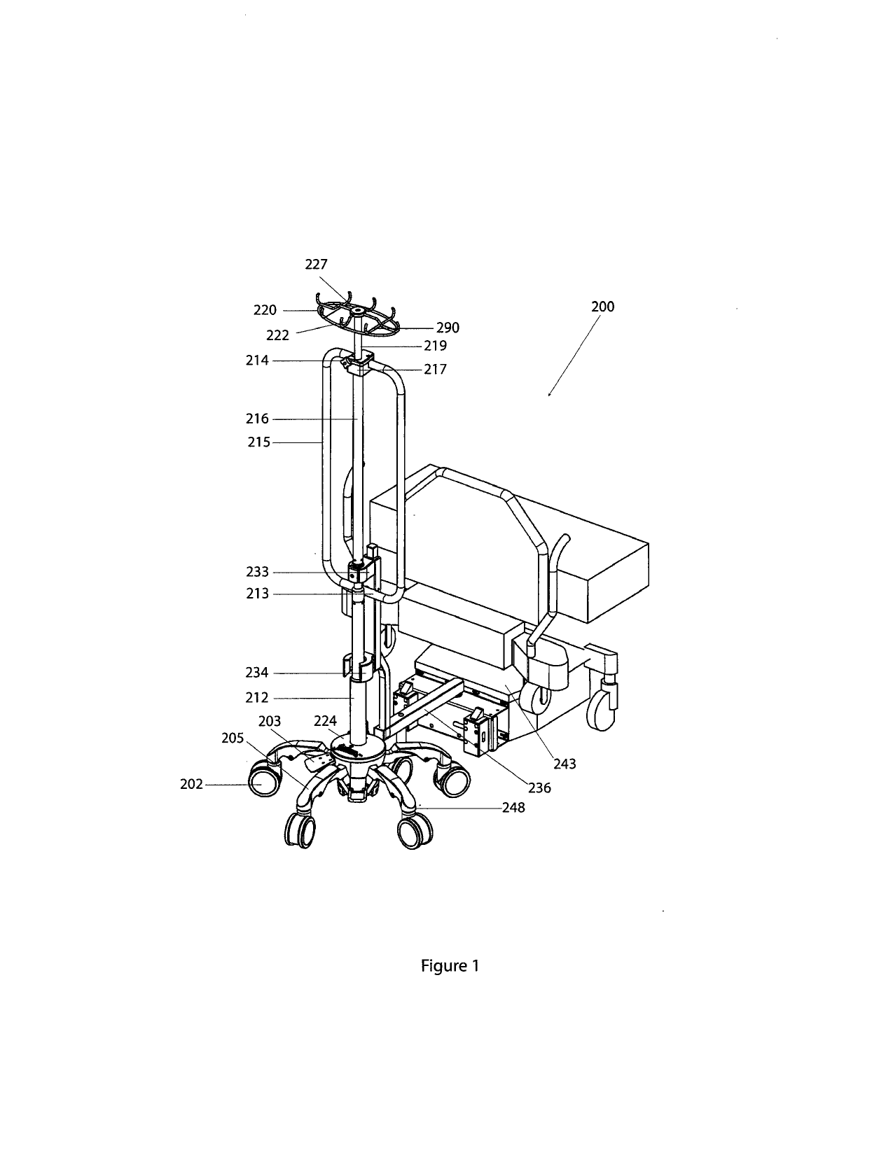

Referring now to FIG. 1, in this example embodiment there is shown an improved retractable IV pole system (200) in which a plurality of legs (205) are in an expanded position. A lifting mechanism (203) allows for non-user powered retraction of a base (224) of an IV pole (201). In at least one embodiment, IV pole (200) comprises base (224) which stabilizes IV pole (200), a top (227) where equipment can be hung, and a main mast (216) extending between base (224) and top (227). FIG. 1 shows IV pole (200) in an expanded state.

In FIG. 1 IV pole system (200) is releasably engaged to a bed mount (243) or other patient holding a transportation apparatus.

Attached at or near the end of each leg (205) is at least one wheel or rolling device (202). In at least one embodiment, the wheels are caster-type wheels capable of freely rotating along a leg-wheel connection (248). Although in this particular depiction there are five legs (205) with five four-inch caster-type wheels (202), embodiments with different numbers of legs (205) (such as 1, 2, 3 or more) and different sized or types of wheels (202) are within the scope of the invention.

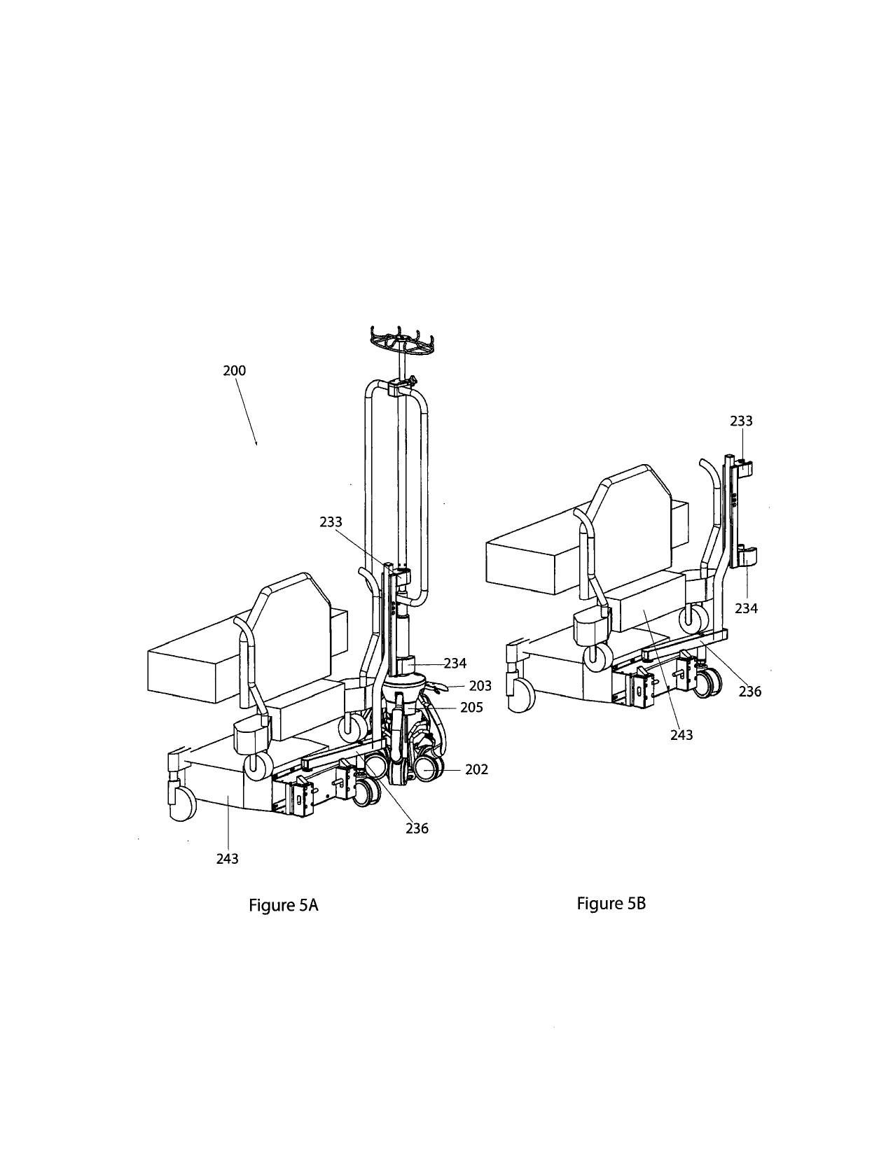

In this example embodiment, base (224) of IV pole system (200) comprises two or more legs (205) which are pivotably engaged to IV pole (200) by leg connections. The leg connections can be pins, screws, nuts or any other pivoting engagement known in the art. When in the expanded state legs (205) are pivoted to extend away from mast (216) of IV pole system (200) to provide a stable support platform. When in the retracted state, shown in FIGS. 5A and 5B, legs (205) are pivoted inwards towards mast (216) of IV pole system (200) allowing IV pole system (200) to be moved with a reduced IV pole system (200) diameter. In a related embodiment, they can retract upwards towards the mast.

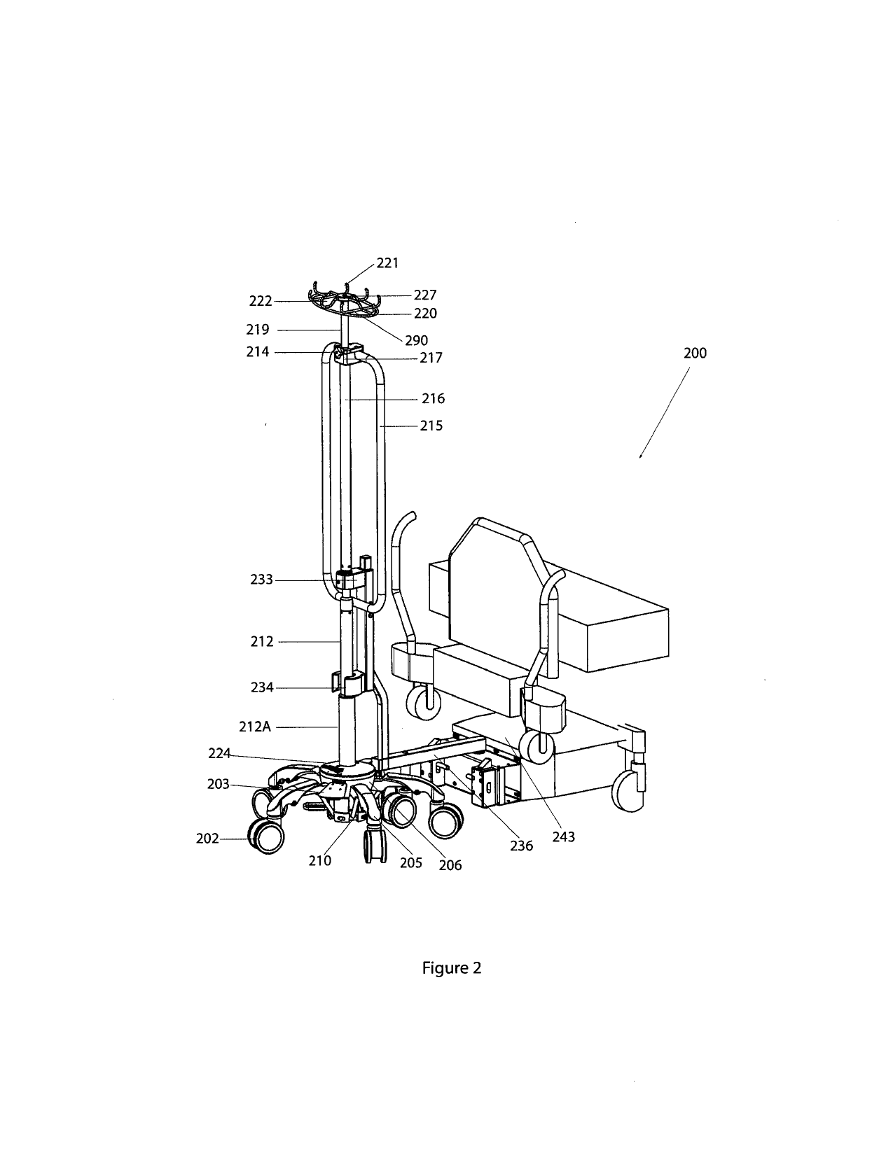

Referring now to FIG. 2, there is shown an embodiment of base (224) of IV pole system (200). A lifting mechanism may be used to retract and expand base (224). One particular lifting mechanism is a gas spring which are well known in the art and are described at least in U.S. Pat. Nos. 7,222,702, 7,073,642, and 4,582,304, all of which are incorporated by reference in their entirety.

In at least one embodiment, IV pole system (200) comprises a base support (206) located at or near the bottom of mast (212). In at least one embodiment, base support (206) is positioned around a portion of mast (212). Base support (206) is one location where leg (205) is pivotably engaged to the IV pole by at least one leg connection (210). As the lifting mechanism (203) initiates the movement of base support (206) upward, the portion of leg (205) near a leg connection (210) is also moved upwards. This upward movement in turn rotates the portion of the leg-wheel connection where the wheel (202) is engaged to the leg (205) into a position closer to the mast (212).

In at least one embodiment, lifting mechanism (203) is integrally related to the base support (206). Some or all of base support (206) can be surrounded at least in part by a housing, which may contain all, or some, of the components which may facilitate the actuation of lifting mechanism (203). In at least one embodiment the housing may also comprise a hygienic fluid tight seal to prevent the entry of liquids, blood, organic matter or other material into the interior of base (224) which may drip or splatter against IV pole system (200) during use.

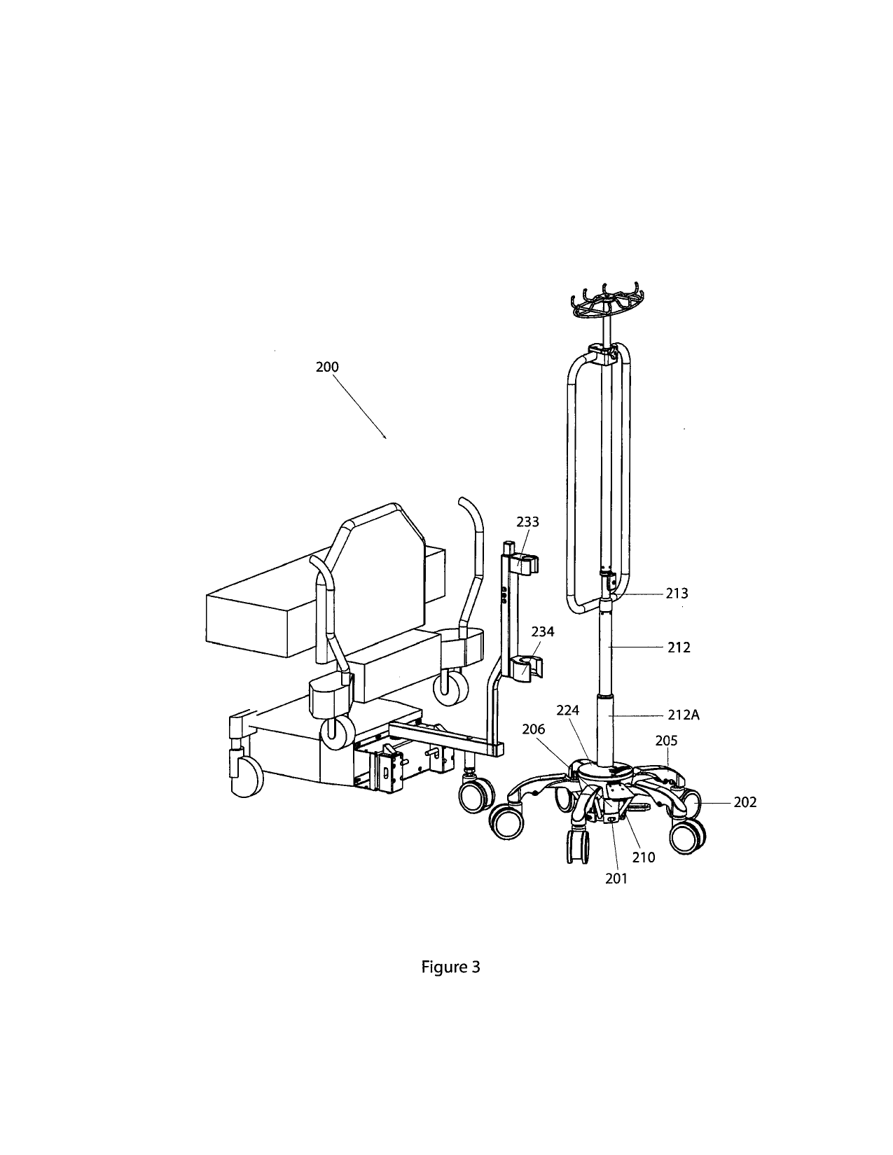

Referring now to FIG. 3, in at least one embodiment, IV pole system (200) also comprises a base center (201) located at the bottom of mast (212) which provides a load bearing bottom that can support the weight of IV pole system (200) in the absence of the wheels (202) or when wheels (202) are retracted. Base center (201) may have a larger diameter than mast (212) in order to provide stability for IV pole (200). Base center (201) however has a narrower diameter than extended legs (205) in order to permit positioning retracted IV pole system (200) closer to a holding apparatus. In at least one embodiment, base center (201) and base support (206) have substantially the same diameter.

In this example embodiment, legs (205) can receive buttressing support from one or more braces or support legs which are engaged to IV pole system (200) and which are also pivotably engaged by an outer connection to legs (205). The leg support, outer connection, or leg support (210) may be located at any desired position along legs (205), so long as the performance of the functional features described herein are not sacrificed.

In at least one embodiment, IV pole system (200) comprises a base sleeve (212A) coupled to base (224). Base sleeve (212A) is a tube or other sleeve arrangement positioned around mast (212) to guide the upward or downward motion of base (224). The sleeve is movable relative to mast (212) and maintains a constant position relative to leg connections (210), base support (206), and/or one of the components of base (224) or any combination thereof. In one embodiment, at least a portion of base sleeve (212A) is at least partially surrounded by the base support (206).

In this example embodiment, IV pole system (200) is adapted for engaging two mounts one being an upper mount (233) and one being a lower mount (234). In at least one embodiment, IV pole system (200) is adapted for engagement by having a narrow portion (213) of the main mast (212) releasably engaged to the upper mount (233). In at least one embodiment, one or more of the mounts have a generally rounded interior with an open side facing IV pole system (200). In at least one embodiment, at least one of the two mounts is a clip which is a biased gripping member (such as a strip of bent metal). All gripping or clipping means known in the art are contemplated by the inventive concept.

Referring to FIGS. 1 and 2 there are shown that in at least one embodiment, upper mast (216) of IV pole system (200) includes one or more wings (215). Mast (216) can be a continuation of bottom portion of mast (212) engaged to base (224) or can be a separate component engaged to mast (212). In this embodiment, wings (215) may be removably attached to upper mast (216). Mast (216) with or without the wings (215) may be used to hold patient equipment including but not limited to IV bags and pumps. Wings (215) also provide additional areas for attachment of patient equipment on IV Pole system (200). In at least one embodiment, at least a portion of mast (216) is hollow and an extending pole (219) may be at least partially housed within the hollow portion. Extending pole (219) is used to raise and support top (227) of IV pole system (200).

As illustrated in FIGS. 1 and 2, in at least one embodiment, near top or upper mast (216) is a mast collar (217) which can be removably placeable at or near top (227) of main mast (216). Mast collar (217) may be set to allow extending pole (219) to be adjustably raised or lowered. In at least one embodiment, a pressing or activating member (214) such as a bolt, screw or pin releasably applies pressure against extending pole (219) thereby either allowing extending pole (219) to be moved higher or lower, or to hold extending pole (219) in place. In at least one embodiment, pressing member (214) includes a knob engaged to the end of pressing member (214) and the holding pressure is applied or released by respectively turning the knob in one circular direction or another. In at least one embodiment, a portion of mast collar (217) surrounds the top of main mast (216), and a portion extends above main mast (216). The portion of mast collar (217) above main mast (216) has an aperture through which pressing member (214) passes through to reach extending pole (219). In at least one embodiment, pressing member (214) passes through apertures in both main mast (216) and mast collar (217). In at least one embodiment, one or more of the wings (215) are engaged to mast collar (217). In at least one embodiment the lower portion of wings (215) are engaged to mast (216) by a connector (213).

In this example embodiment, top (227) of IV pole system (200) includes a halo (220) engaged to adjustable extending pole (219). Adjustable extending pole (219) allows for halo (220) to be positioned at the optimal height for hanging items of medical equipment. In at least one embodiment, halo (220) is a ring reinforced by one or more support bars (222) which radiate from an engagement point on extending pole (219) out to the ring. In at least one embodiment, support bars (222) are reinforced by crossbars (290) that extend from one point on the ring to another and supportively intersect t support bars (222). One or more of halo (220), halo support bars (222), and/or halo crossbars (290) can be constructed out of metal. Halo (220) can be of any shape including but not limited to circular, elliptical, ovoid, rounded, angular, curved, square, rectangular, triangular, trapezoidal and any combination thereof.

In this example embodiment, positioned around halo (220) are halo hooks (221) which may also be constructed out of metal. Halo hooks (221) may hold medical equipment or accessories including but not limited to patient fluid bags, IV bags, other medicine bags and any combination thereof. Halo hooks (221) can be curved as illustrated in FIGS. 1 and 2 or can be of any other hooking shape. In addition, halo hooks (221) can be extensions of halo support bars (222) or can be circumferentially offset from halo support bars (222).

In an example embodiment IV Pole System (200), having a lifting mechanism for retracting and extending a plurality of legs. The IV pole system (200) provides a user a means of engaging a lifting mechanism associated with the base and the mast. When the lifting mechanism is engaged, the lifting mechanism initiates movement of the base member along the mast.

In addition to using a gas spring to retract the legs, this invention contemplates various embodiments in which the legs are retracted by any lifting mechanism known in the art including but not limited to hydraulic devices, electrical motors, coil springs, gears, hand cranks, magnetic repulsion devices and any combination thereof. Contemplated embodiments envision a lowering mechanism as well which extends the legs through the lifting mechanism operating in reverse or through the use of another pushing mechanism including but not limited to gas springs, hydraulic devices, electrical motors, coil springs, gears, hand cranks, magnetic repulsion devices and any combination thereof. The lifting mechanism and lowering mechanism may also be combined to a singular mechanism.

One embodiment of the invention is directed to an IV pole system for supporting medical equipment comprising: a base having at least one leg, a mast engaged to the base, and a lifting mechanism. The at least one leg may comprise at least one wheel. The mast is engaged to the base and comprises a length, a top and a bottom.

The lifting mechanism may comprise a component to provide a more easy retracting and expanding of the at least one leg. These components are selected from a group consisting of, but not limited to, a gas spring, a hydraulic device, an electrical motor, a mechanical gear assembly, a coil spring, a hand crank and magnetic repulsion devices. The lifting mechanism is constructed and arranged to position at least one leg in either an extended configuration or an in a retracted configuration. In one example embodiment, at least a portion of the wheel is disposed lower than the bottom of the mast when the leg is in the extended configuration. The wheel is radially separated from the mast in the extended configuration. In various embodiments, the lifting mechanism is further constructed and arranged to elevate the base and elevate at least a portion of the leg proximate to the mast in the retracted configuration and to position the wheel radially proximate to the mast in the retracted configuration.

At least one embodiment of the invention is directed to an IV pole in which the gas spring comprises two ends, a cylinder with a pin aperture and a plunger. The plunger and the cylinder are movably engaged to each other. The gas spring is constructed and arranged to separate the two ends and to alter the length of the mast. The cylinder comprises a compressible fluid and is constructed and arranged to exert a pneumatic force on the plunger to separate the two ends. The cylinder is further constructed and arranged to exert a pneumatic force on the plunger to elevate the base and to position at least one leg into the retracted configuration.

At least one embodiment of the invention is directed to an IV pole having a leg that is pivotably engaged to the mast. A brace can be pivotably engaged to the base and at least one leg. The brace having a pivotal engagement is elevated relative to the leg's pivotal engagement when the leg is in the retracted configuration.

At least one embodiment of the invention is directed to an IV pole in which the gas spring cylinder is proximal to the base. The plunger can be proximal to the top of the mast while the gas spring can be positioned within a hollow portion of the mast. The IV pole can also have a gas release pin which is constructed and arranged to be inserted into and separated from a pin aperture and to actuate the gas spring. A pedal having a pedal lever can be engaged to the gas release pin. The pedal lever can be constructed and arranged to insert and separate the gas release pin relative to the pin aperture.

At least one embodiment of the invention is directed to an IV pole further comprising at least one wing extending substantially parallel to the mast and being engaged to the mast. The wing may be removably engaged to the mast. A halo may be engaged to the mast proximate to the top. The halo comprises an outer frame having at least one hook projecting away from the frame.

At least one embodiment of the invention is directed to an IV pole system further comprising a pole mount. The pole mount comprises a horizontally sliding bar engaged to both a vertically extending support bar and at least one engagement member. The vertically extending support bar comprises an elevating mechanism and one or more gripping members. The gripping members are constructed and arranged for engagement to the mast. The lifting mechanism is constructed and arranged to adjust a vertical position of the mast. The horizontally sliding bar is constructed and arranged to adjust a horizontal separation distance between the at least one engagement member and the vertically extending support bar. The at least one engagement member can be constructed and arranged to engage a hospital bed. A bumper tail can be positioned around at least a portion of the bed. The bumper rail bulges farther from the bed at bed corners than from other bed locations. The system further comprises a support bar engaged to the elevating member which is constructed and arranged to lift the bar. A biasing mechanism can exert a force on the horizontally sliding bar reducing the separation distance between the at least one engagement member and the vertically extending support bar. The horizontally sliding bar can have an inner portion in a telescoping relationship to the outer portion. The horizontally sliding bar can be pivotably engaged to the at least one engagement member.

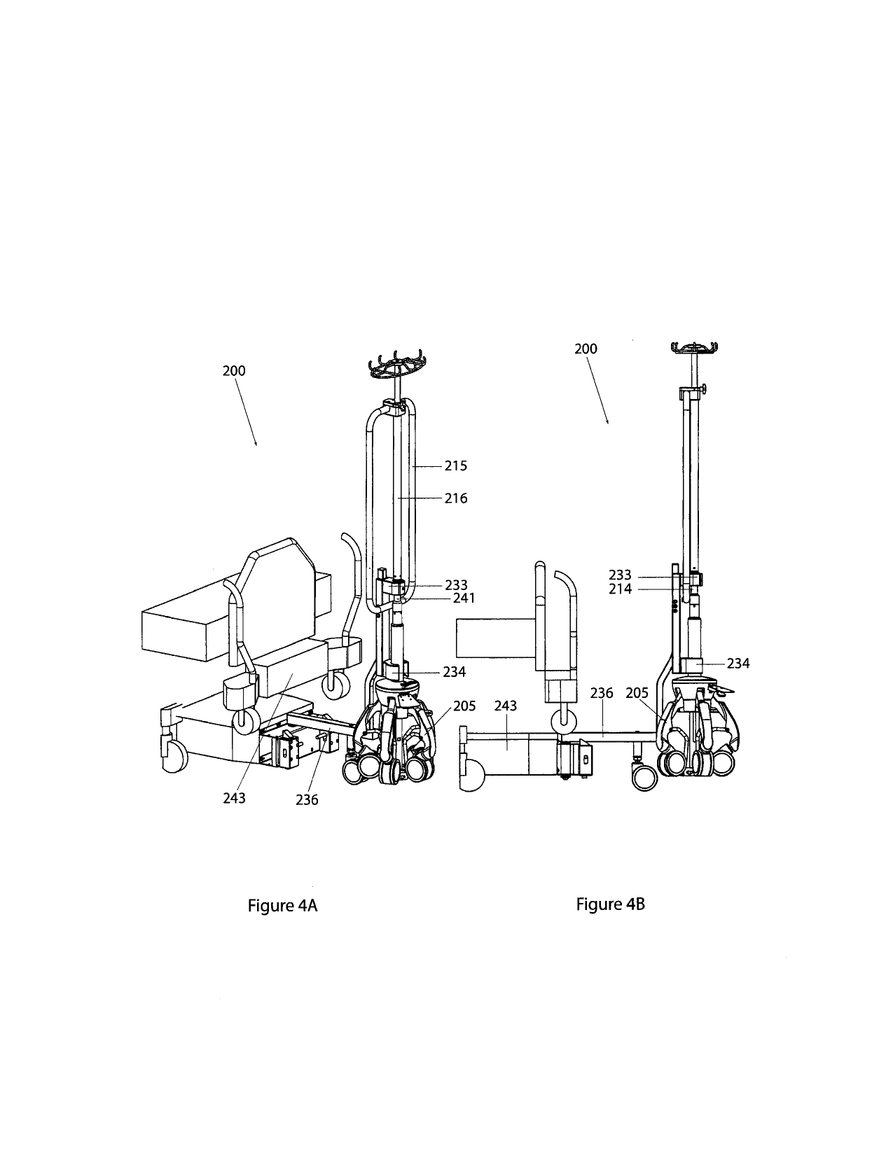

Referring now to FIGS. 4A and 4B, it is shown, in this example embodiment, that IV pole system (200) can be wheeled towards mounts (233, 234) and a narrow portion (214) slides within or adjacent to upper mount (233). In at least one embodiment, one or more supplemental masts or wings (215) descend lower than narrow portion (214) allowing lower placement of equipment on wings (215) than on main mast (216). In at least one embodiment, portions above and below narrow portion (214) gradually widen to the width of main mast (216). These tapered portions allow IV pole system (200) to be positioned above or below mounts (233, 234) and appropriately drop or ascend into proper position. Use of tapered portions allows the user to secure IV pole system (200) to mounts (233, 234) without needing to perfectly align narrow portion (214) with mounts (233, 234).

Main mast (216) is supported by the mounts (233, 234) and in at least one embodiment, upper mount (233) is constructed and arranged to grasp the diameter of narrow portion (214) of main mast (216). In at least one embodiment, lower mount (234) is constructed and arranged to grasp the diameter of the bottom of mast (212) or base sleeve. In at least one embodiment, lower mount (234) has a clip or clamp or other portion sized to grasp the diameter of mast (212) or base sleeve. Mount (233, 234) may also include a mechanism to adjust the height of the mount. This adjusting means provides for IV pole system (200) to be easily coupled with at least one of the mounts (233 and/or 234).

In at least one embodiment, IV pole system (200) is suspended in the air or at least supported prior to engaging lifting mechanism (203). By suspending or supporting IV pole system (200) prior to actuation, at least some of the weight of IV Pole system (200), is removed from the wheels (202) which facilitates a more efficient retraction of legs (205) of IV pole system (200). In a related embodiment, the weight is relieved by rolling IV Pole system (200) into at least one or more mounts (233, 234) which lift IV pole system (200) and bear the weight of IV pole system (200) and equipment.

In at least one embodiment, IV pole system (200) comprises an internal spring that applies continuous pressure on the legs prior to engaging lifting mechanism (203). In a related embodiment, the internal spring that applies continuous pressure on the legs is absent. In at least this embodiment, some of the weight of IV Pole system (200) is removed from the wheels (202) which facilitates a more efficient retraction of legs (205) of IV pole system (200). The IV pole system (200) base support (206) is positioned above the ground surface and act as the support mechanism when IV support system (200) is not engaged in a mounting means. In a related embodiment, the weight may be also relieved by rolling IV Pole system (200) into one or more mounts (233, 234) which lift IV pole system (200) and bears the weight of IV pole system (200) and equipment.

Referring still to FIGS. 4A and 4B, in at least one embodiment, when IV pole system (200) is properly positioned within mounts (233, 234), as legs (205) retract, the bottom portion of the mast slides upward and into lower mount (234). This secures IV pole system (200) from easily moving out of the lower mount (234). By this method, legs (205) can be securely positioned close to a patient holding or transportation apparatus with little effort by the user. In at least one embodiment, all the user needs to do is roll IV pole system (200) into a desired location relative to a patient transport apparatus and activate lifting mechanism (203) to ready IV pole (200) for easy transport. FIGS. 4A and 4B depicts IV pole (200) in its retracted configuration and is off of the ground after it has already been mounted onto bed mount (243). In at least one embodiment, IV pole system (200) suspended by mounts (233, 234) lifts IV pole system (200) off the ground before activating the gas spring. In a related example embodiment, pole (200) is supported by one clamp or mount (233 or 234).

FIGS. 5A and 5B show that in at least one embodiment, after which IV pole system (200) is secured by mounts (233, 234), the operator presses lifting mechanism (203), being in mechanical communication with base support (206), and raises legs (205) off the ground. Lifting IV pole system (200) off the ground reduces or eliminates any hysteresis effects and assures that lifting mechanism (203) easily retracts legs (205). In at least one embodiment, mounts (234, 233) are in movably engaged in communication with a holding apparatus such as a bed. In at least one embodiment, horizontal support bar (236) rotates via mechanical communication with a mount bar pivot. The rotatable communication allows for movement around the bed. This movement is important when maneuvering a bed and IV pole system (200) through tight areas such as, but not limited to, hallways, rooms, and elevators. In order to allow for a smooth 180 degree rotation around the bed, bed mount (243) (which can be mounted on the head of a bed) can be positioned around the perimeter of at least a portion of the bed. The bumpers prevent the rotating vertical support portion of bed mount (243) from catching any point of the bed. Additional bed mount components are further described in co-owned U.S. Pat. No. 7,918,422, which is incorporated herein by reference in its entirety. In a related embodiment, IV pole system (200) is integrally mounted directly to a bed or patient transportation device.

In at least one embodiment, legs (205) are outwardly extended during elevation of IV pole (200) by mounts (233, 234). In at least one embodiment, legs (205) are manually pushed down by the user prior to release from mount (233, 234). In at least one embodiment, IV pole (200) “pops” off of mounts (233, 234) with the application of a pushing force by the operator in a direction opposite that used to position IV pole (200) within mounts (233, 234). In at least one embodiment, all of the components which couple an IV pole (200) to a bed disclosed in FIGS. 1-5 are adapted for use on a wheel chair or other patent transport device. In at least one embodiment, as the legs are pushed down they contact the ground and then exert a lifting force against mast (216). This lifting force lifts mast (216) out of mounts (234, 233) thereby releasing IV pole system (200).

The above disclosure is intended to be illustrative and not exhaustive. This description will suggest many variations and alternatives to one of ordinary skill in this art. The various elements shown in the individual figures and described above may be combined, substituted, or modified for combination as desired. All these alternatives and variations are intended to be included within the scope of the claims where the term “comprising” means “including, but not limited to”.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claims below.

We claim:

1. A method of retracting and extending a plurality of legs of a base member of an IV pole system, the IV pole system having a mast engaged to said base member and having a length, a top and a bottom, the method comprising:
engaging a lifting mechanism associated with the base and the mast, wherein said lifting mechanism initiates movement of the base member along the mast.

2. The method of claim 1, wherein the step of engaging said lifting mechanism includes initiating movement of retracting the plurality of legs.

3. The method of claim 1, wherein the step of engaging said lifting mechanism includes initiating movement of extending the plurality of legs.

4. The method of claim 2, wherein the IV pole system retracted position has a base member that supports said legs being above a surface.

5. The method of claim 2, wherein the IV pole system retracted position has the base member with said legs being at least partially above the bottom of the mast.

6. The method of claim 2, wherein the IV pole system retracted position has the base member with said legs being at least partially below the bottom of the mast.

7. The method of claim 1, wherein the lifting mechanism includes a component selected from a group consisting of a gas spring, a hydraulic device, an electrical motor, a mechanical gear assembly, a coil spring, a hand crank and magnetic repulsion devices.

8. The method of claim 1, wherein each of the legs includes at least one rolling device.

9. The method of claim 1 further including disposing a portion of said mast in a means for mounting.

10. The method of claim 9 wherein disposing said mast further includes releasably engaging a portion of said mast in said mounting means.

11. The method of claim 9, wherein disposing said mast further includes releasably disengaging a portion of said mast from said mounting means.

12. The method of claim 9, wherein disposing said mast in said mounting means includes lifting the mast to said mounting means such that the bottom of the mast is above a surface on which the IV pole system is placed.

13. The method of claim 9, wherein said mounting means includes a means for raising or lowering said mounting means.

14. The method of claim 13, wherein said mounting means being integrally related to a bed or patient transportation apparatus.

15. The method of claim 9, wherein said mounting means being integrally related to a bed or patient transportation apparatus.

16. A method of retracting and extending a plurality of legs of a base member of an IV pole system, the IV pole system having a mast engaged to said base member and having a length, a top and a bottom, the method comprising:
actuating a plurality of legs associated with the base member of the mast, said actuating includes providing movement of said plurality of legs about the mast.

17. The method of claim 16, wherein the step of said actuating includes initiating movement of retracting the plurality of legs.

18. The method of claim 16, wherein the step of said actuating includes initiating movement of extending the plurality of legs.

19. The method of claim 17, wherein the IV pole system in a retracted position has a base member that supports said legs being above a surface.

20. The method of claim 17, wherein the retracted position has a base member with said legs being above the bottom of the mast.

21. The method of claim 17, wherein the retracted position has a base member with said legs being below the bottom of the mast.

22. The method of claim 16, wherein actuating includes engaging a component selected from a group consisting of a gas spring, a hydraulic device, an electrical motor, a mechanical gear assembly, a coil spring, a hand crank and magnetic repulsion devices.

23. The method of claim 16, wherein each of the legs includes at least one rolling device.

24. The method of claim 16 further including disposing a portion of said mast in a means for mounting.

25. The method of claim 24 wherein disposing said mast further includes releasably engaging a portion of said mast in said mounting means.

26. The method of claim 24 wherein disposing said mast further includes releasably disengaging a portion of said mast from said mounting means.

27. The method of claim 24, wherein disposing said mast in said mounting means includes lifting the mast to said mounting means such that the bottom of the mast is above a surface on which the IV pole system is placed.

28. The method of claim 24, wherein said mounting means includes a means for raising or lowering said mounting means.

29. The method of claim 28, wherein said mounting means being integrally related to a bed or patient transportation apparatus.

30. The method of claim 24, wherein said mounting means being integrally related to a bed or patient transportation apparatus.

31. An IV pole system for supporting medical equipment comprising:
a base member having a plurality of legs,
a mast engaged to said base member said mast comprising a length, a top, and a bottom, and
a lifting mechanism configured and arranged to move the base member along the mast.

32. The IV pole system of claim 31, wherein the base member is adapted to be responsive to the lifting mechanism to position said plurality of legs in an extended configuration.

33. The IV pole system of claim 31, wherein the base member is adapted to be responsive to the lifting mechanism to position said plurality of legs in a retracted configuration.

34. The IV pole system of claim 31, wherein the lifting mechanism includes a component selected from a group consisting of a gas spring, a hydraulic device, an electrical motor, a mechanical gear assembly, a coil spring, a hand crank, and magnetic repulsion devices.

35. The IV pole system of claim 31, wherein at least one of said plurality of legs includes at least one rolling device.

36. The IV pole system of claim 31, wherein each of said plurality of legs includes at least one rolling device.
